(19) United States
(12) Patent Application Publication
(10) Pub. No.: US 2009/0169442 A1
(43) Pub. Date: Jul. 2, 2009

(54) ANTI-DEPOSIT COATING ON INTERNAL SURFACES OF AN ULTRAVIOLET DISINFECTION SYSTEM

(76) Inventors: Uri LEVY, Rehovot (IL); Zohar Vardiel, Neve Savyon (IL)

Correspondence Address: Pearl Cohen Zedek Latzer, LLP, 1500 Broadway, 12th Floor, New York, NY, 10036, US

(21) Appl. No.: 12/347,092

(22) Filed: Dec. 31, 2008

Related U.S. Application Data
(60) Provisional application No. 61/017,811, filed on Dec. 31, 2007.

(51) Int. Cl. A61L 2/10; B05D 5/08; B32B 27/06

(52) U.S. Cl. 422/186.3; 427/160; 427/230; 427/239; 428/421; 428/422

(57) ABSTRACT

Embodiments of the invention are directed to a system and a method for reducing deposit formation in an ultraviolet (UV) liquid disinfection system by applying a coating layer of a flouropolymeric anti-deposit material on a surface of the UV disinfection system.

### CROSS REFERENCE TO RELATED APPLICATIONS

This application claims benefit of U.S. Provisional Application No. 61/017,811, filed on Dec. 31, 2007 which is incorporated in its entirety herein by reference.

### BACKGROUND OF THE INVENTION

Disinfection systems using ultraviolet (UV) light have been long known. A major problem, which reduces the effectiveness of UV water treatment, is the formation of deposit on interior surfaces of the UV reactor and in particular on UV transmissive surfaces, such as quartz sleeves. The deposit results from dissolved chemical materials and organic matter entities existing in the untreated liquid precipitating onto or sticking to surfaces contacting the liquid. Such surfaces are the reactor's interior walls and external surfaces of quartz sleeves protecting the UV lamps. The formation of a film or biofilm onto UV transmissive surfaces, such as quartz sleeves, UV optical windows or walls reduce the transmissiveness and and/or reflective properties of these surfaces. Such reduction in optical properties may be translated into reduction in reactor performance.

A variety of approaches have been provided to overcome this problem. A first known approach has been to periodically interrupt the disinfection process for maintenance operations which includes mechanical removal of the deposit film, manual cleaning of the surfaces and/or replacement of dirty parts. Such a maintenance period is an undesirable, expensive and time-consuming procedure. Another approach has been to use chemicals for cleaning the reactor. The use of chemicals for cleaning, which also requires interrupting the disinfection operation, is most undesirable for economic considerations and environmental considerations. An integral and efficient solution which may increase the period between successive interruptions or even eliminate interruptions altogether is highly desired.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a cross section illustration of a UV disinfection system having anti-deposit coating according to some embodiments of the present invention;

FIGS. 2A and 2B are conceptual illustrations of another disinfection system having anti-deposit coating according to some embodiments of the invention; and

FIG. 3 is an illustration of another UV disinfection system having anti-deposit coating according to some demonstrative embodiments of the invention;

It will be appreciated that for simplicity and clarity of illustration, elements shown in the drawings have not necessarily been drawn accurately or to scale. For example, the dimensions of some of the elements may be exaggerated relative to other elements for clarity. Further, where considered appropriate, reference numerals may be repeated among the drawings to indicate corresponding or analogous elements. Moreover, some of the blocks depicted in the drawings may be combined into a single function.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits may not have been described in detail so as not to obscure the present invention.

Some demonstrative embodiments of the invention include coating interior surfaces of a UV reactor used for UV liquid disinfection with an anti-deposit coating material, such as Teflon®. It should be understood that other materials having similar anti-sticking and UV-transparency properties may be used. Embodiments of the present invention are directed to a system and method for preventing and/or reducing deposit formation on interior surfaces of disinfection systems. According to some embodiments of the present invention, a coating layer made of anti-deposit material may be used in order to prevent or reduce the build-up of deposit on interior surfaces of a reactor of a UV disinfection system.

Although embodiments of the present invention are described as directed to disinfection systems, it should be understood to a person skilled in the art that embodiments of the present invention may be used in a variety of applications, systems and devices which may include disinfection of flowing or non-flowing liquid. For example, embodiments of the present invention may be used in water reservoirs, aquariums, portable UV reactors or any other water-contained tanks.

Embodiments of the present invention make use of fluoropolymers having non-stick anti-adhesive characteristics such as, PTFE (polytetrafluoroethylene) commonly known as Teflon®, PFA (perfluoroalkoxy polymer resin), FEP (fluorinated ethylene-propylene) and the like. Other fluoropolymers may include polyethylenetetrafluoroethylene (ETFE), polyvinylfluoride (PVF) and PFPE Perfluoropolyether. Also, they are stable and tend to be chemically inert. Fluoropolymers may be mechanically characterized as thermosets or thermoplastics.

The fluoropolymers referred to collectively as “Teflon” and also referred to herein as “anti-deposit materials”, may be used as a coating layer in UV-based liquid disinfection systems to prevent or reduce deposit buildup on internal surfaces contacting the liquid due to their unique characteristics.

Thin Teflon (PTFE) layers (0.01 to 0.1 mm) are essentially transparent to U light. Accordingly, the optical properties of the transparent surfaces or elements are essentially maintained when coated with Teflon. For example, the ability of a quartz sleeve surrounding a UV lamp to pass on the UV light emitted from the lamp remains substantially unaffected when coated with Teflon. Transmission tests for uncoated vs. Teflon coated quartz sleeves have shown the transmission of UV light at a wavelength of 254 nm for the coated quartz to be 95% of the transmission of the uncoated quartz sleeve at similar conditions.

Further, Teflon is known to be a UV-resistant material essentially retains its original properties over time when exposed to UV light. A durability test has shown that, after 24 hours of emitting UV light at a wavelength of 254 nm from a 110 W/cm medium pressure UV lamp enclosed by a Teflon-coated quartz sleeve immersed in flowing water, the transmission was not degraded.

Other desired characteristics of a material suitable as an anti-deposit material for UV liquid disinfection systems may be bio compatibility, strong water-repellency and ability to withstand high temperature as well as smoothness and anti-adhering characteristics.

It should be understood to a person skilled in the art that the coating layer on some portions of the surface or some elements may be identical or different to the coating layer on other portions of the surface or other elements. The desired thickness of the coating layer may be determined so as to ensure the desired mechanical, optical and chemical properties of the coating including adhesion to the surface, UV transmission and durability. According to embodiments of the invention, the thickness of the coating layer may be between 10-50 micron. According to other embodiments of the invention, the thickness of the coating layer may be above 50 micron. The thickness of the coating layer may vary as needed, for example, it may not be uniform for the entire coated areas.

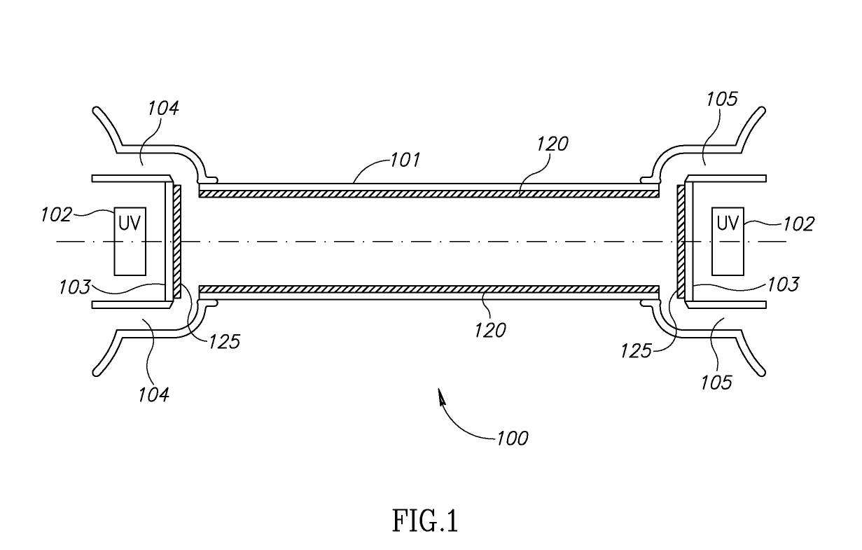

Embodiments of the present invention may be applicable in a plurality of UV disinfection systems having variety of designs, shapes, size and/or other characteristics. Although the present invention is not limited in this respect, some exemplary designs of disinfection systems utilizing an anti-deposit coating layer according to embodiments of the invention are illustrated in FIGS. 1-3 below. It should be understood to a person skilled in the art that embodiments of the present invention may be used in any other flowing or standing liquid disinfecting systems.

Reference is now made to FIG. 1 which is a cross section illustration of a disinfection system having anti-deposit coating according to some embodiments of the present invention. The anti-deposit coating may prevent or reduce deposit formation on various elements, parts or areas of the disinfection system contacting the liquid. According to some embodiments of the invention, a disinfection system 100 may include a conduit 101 to carry flowing liquid to be disinfected and one or more external UV sources 102 to illuminate and to disinfect the liquid within conduit 101. Conduit 101 may have an inlet 104 to receive the liquid, and an outlet 105 to discharge the liquid. Conduit 101 may be made, at least partially, of a UV transparent material, such as quartz. Conduit 101 may include one or more elements made of a UV transparent material. Conduit 101 or one or more elements of conduit 101 may be internally covered or coated at least partially, with an anti-deposit layer 120, such as Teflon layer.

Disinfection system 100 may include one or more windows 103 which may be made of UV transparent material, such as quartz and may be located at one or more ends of conduit 101, proximate to illumination source 102. Windows 103 may be covered or coated on the surface contacting the liquid at least partially, with anti-deposit material layer 125, such as Teflon layer.

According to some embodiments of the invention, anti-deposit layers 120 and 125 may be transparent to UV light and as such may maintain the optical properties of the surfaces underneath. For example, a conduit made of quartz and coated with anti-deposit layer 120 may act as a waveguide and at least part of the UV light emitted from illumination source 102 and entering conduit 101 may be totally-internally reflected at the interface of the UV-transparent conduit and the air surrounding it. In another example, a conduit made of metallic material may be coated with a reflective coating coated with anti-deposit layer 120 and may reflect UV light back into the water.

Being made of a fluoropolymer, such as Teflon, anti-deposit coating layers 120 and 125 may have additional desired characteristics including UV-resistivity, water-resistivity and high temperatures-resistivity which may all contribute to the durability and stability of the anti-deposit coating layers. In addition, being made of fluoropolymers, coating layers 120 and 125 may be bio-compatible and non-toxic and as such may as well, be suitable to be used in water disinfecting systems.

Further, the use of fluoropolymeric materials such as PTFE, PFA, FEP for coating internal surfaces of water disinfection systems may enable the use of otherwise non-acceptable materials, such as aluminum to create internal UV-reflective surfaces. The conduit may include internal aluminum sheets coated with the anti-deposit material. The coating layer may serve as a barrier between the aluminum surface and the liquid without affecting the optical properties of the aluminum.

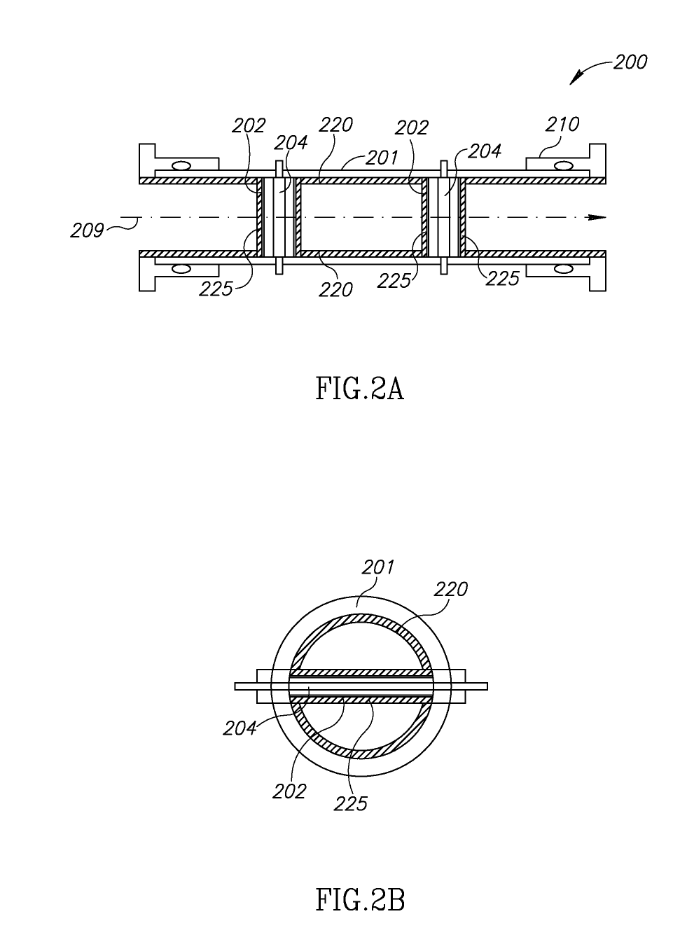

Reference is now made to FIGS. 2A and 2B, which conceptually illustrate a disinfection system having anti-deposit coating according to some demonstrative embodiments of the invention. A disinfection system 200 may include a conduit 201 made of UV-transparent material. Such as quartz to carry liquid to be disinfected, one or more UV-transparent sleeves 202 positioned within conduit 201 substantially perpendicular to its longitudinal axis of symmetry 209 and one or more UV light sources 204, each positioned within a respective sleeve 202.

Disinfection system 200 may include one or more an anti-deposit coating layers on at least part of the interior surface of conduit 201 to prevent or reduce deposit formation on various elements, parts or areas in the interior of conduit 201. According to embodiments of the invention, conduit 201 may be coated, at least partially, with an anti-deposit layer 220, such as Teflon layer and sleeves 202 may be covered with an anti-deposit layer 225, such as Teflon layer in order to substantially prevent deposit formation on conduit 201 and sleeves 202.

As may be seen in FIG. 2B, each sleeve 202 may have external dimensions smaller than the internal dimensions of conduit 201 such that liquid may flow within conduit 201 around sleeves 202. Both ends of sleeve 202 may extend from the walls of conduit 201 to enable replacement of light source 204 within sleeve 202. UV light sources 204 may illuminate the liquid to be disinfected when flowing in the conduit via anti-deposit layer 225. In this configuration, the liquid within conduit 201 may act as a waveguide and at least part of the light emitted from the UV light source may be totally-internally reflected at the interface of conduit 201 coated with anti-deposit layer 220 and the air surrounding it.

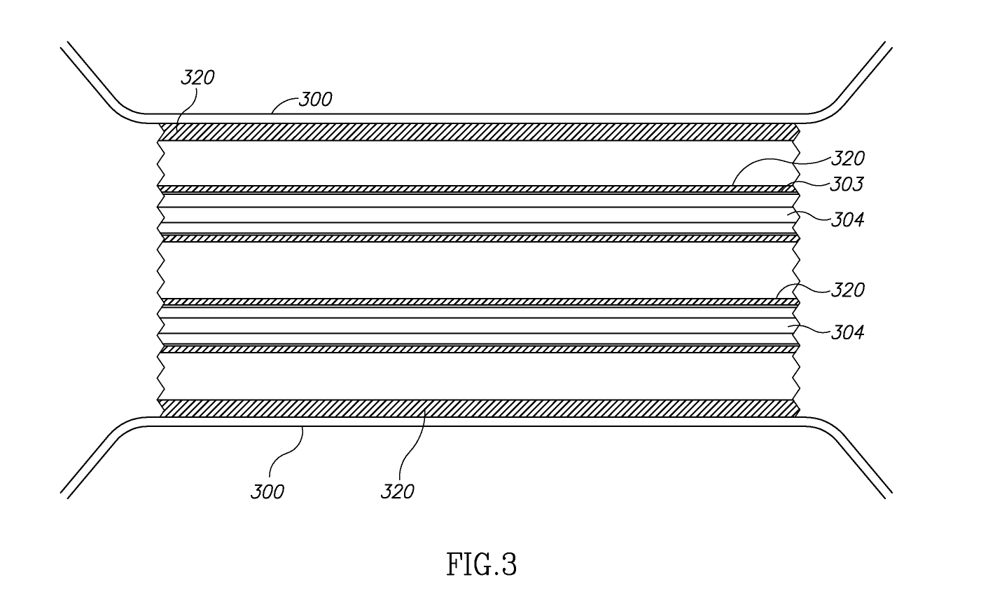

Reference is now made to FIG. 3, which conceptually illustrate a disinfection system having anti-deposit coating according to some demonstrative embodiments of the invention. According to embodiment of the present invention any UV-based disinfecting system, for example, a conventional UV reactor 300 may include an anti-deposit coating layer 320 on internal surfaces, parts or elements, such as sleeves 303 to prevent or reduce deposit formation on the coated portions within the interior of reactor 300 on surfaces contacting the liquid.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. A method for reducing deposit formation in an ultraviolet (UV) liquid disinfection system, the method comprising:
applying a coating layer of a flouropolymeric anti-deposit material on a surface of the UV disinfection system intended to be in contact with liquid to reduce the rate of deposit formation on the surface.

2. The method of claim 1, wherein the surface is an internal surface of a conduit carrying the liquid.

3. The method of claim 2, wherein the internal surface is an aluminum surface.

4. The method of claim 2, wherein the internal surface is transparent to UV.

5. The method of claim 1, wherein the surface is a surface of a protective sleeve that surrounds a radiation source and positioned within a conduit carrying the liquid.

6. The method of claim 1, wherein the anti-deposit material is polytetrafluoroethylene (PTFE).

7. The method of claim 1, wherein the anti-deposit material is perfluoroalkoxy polymer resin or fluorinated ethylene-propylene.

8. The method of claim 1, wherein coating layer has a thickness of about 0.01 to 0.1 millimeters.

9. An ultraviolet (UV) disinfection system comprising:
a conduit to carry liquid to be disinfected;
at least one UV source to illuminate the liquid with UV light; and
a coating layer of a flouropolymeric anti-deposit material on a surface of the UV disinfection system intended to be in contact with liquid to reduce the rate of deposit formation on the surface

10. The system of claim 9, wherein the conduit includes UV transparent walls and the anti-deposit material is applied to the UV transparent walls.

11. The system of claim 9, wherein the conduit includes metal walls and the anti-deposit material is applied to the metal walls.

12. The system of claim 9, wherein the conduit includes at least one internal aluminum sheet having a UV-reflective surface and the anti-deposit material is applied to the UV-reflective surface.

13. The system of claim 9, comprising a UV-transparent protective sleeve to protect the UV source and the anti-deposit material is applied to an external surface of the protective sleeve.

14. The system of claim 9, wherein the anti-deposit material is polytetrafluoroethylene (PTFE).

15. The system of claim 9, wherein the anti-deposit material is perfluoroalkoxy polymer resin or fluorinated ethylene-propylene.

16. The system of claim 9, wherein the coating layer has a thickness of about 0.01 to 0.1 millimeters.

17. The system of claim 9, wherein the coating layer is transparent to UV light.

18. The system of claim 10, wherein at least part of the UV light is totally-internally reflected at the UV transparent walls.

19. The system of claim 9, wherein the UV source is located externally to the conduit and the UV light from the UV source enters the conduit through a UV transparent window coated with the anti-deposit material.
